(19) United States
(12) Patent Application Publication
(10) Pub. No.: US 2016/0256693 A1
(43) Pub. Date: Sep. 8, 2016

(54) SYSTEMS, DEVICES, AND METHODS FOR ELECTRICAL STIMULATION USING A CHEMICAL BIOMARKER FOR FEEDBACK TO ADJUST STIMULATION PARAMETERS

(71) Applicant: Boston Scientific Neuromodulation Corporation, Valencia, CA (US)

(72) Inventor: Jordi Parramon, Valencia, CA (US)

(21) Appl. No.: 15/057,862

(22) Filed: Mar. 1, 2016

Related U.S. Application Data
(60) Provisional application No. 62/129,588, filed on Mar. 6, 2015.

(51) Int. Cl. A61N 1/36

(52) U.S. Cl. CPC A61N 1/36139; A61N 1/36071

(57) ABSTRACT

An electrical stimulation system includes a chemical biomarker sensor to monitor, measure, or observe a chemical biomarker in a body of a patient; a control module to use stimulation parameters to provide electrical stimulation signals to an electrical stimulation lead coupled to the control module for stimulation of patient tissue; and a processor to communicate with the chemical biomarker sensor to generate an adjustment to one or more of the stimulation parameters based on the monitoring, measurement, or observation of the chemical biomarker by the chemical biomarker sensor. One example of a chemical biomarker sensor is an adenosine sensor.

### CROSS-REFERENCE TO RELATED APPLICATIONS

This application claims the benefit under 35 U.S.C. §119(e) of U.S. Provisional Patent Application Ser. No. 62/129,588, filed Mar. 6, 2015, which is incorporated herein by reference.

### FIELD

The present invention is directed to the area of implantable electrical stimulation systems and methods of making and using the systems. The present invention is also directed to implantable electrical stimulation systems that include devices or methods for electrical stimulation which utilize a chemical biomarker to provide feedback for adjusting stimulation parameters, as well as methods of making and using the electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One embodiment is an electrical stimulation system including an electrical stimulation lead configured and arranged for implantation in a body of a patient; a control module coupleable to the electrical stimulation lead and including an antenna and a processor coupled to the antenna, where the control module is configured and arranged to provide electrical stimulation signals to the electrical stimulation lead for electrical stimulation of patient tissue; an external programming unit configured and arranged to communicate with the processor of the implantable control module using the antenna and to provide or update stimulation parameters for production of the electrical stimulation signals; and a chemical biomarker sensor configured and arranged to monitor, measure, or observe a chemical biomarker in response to the electrical stimulation.

In at least some embodiments, the control module is configured and arranged for implantation in the body of the patient. In at least some embodiments, the system also includes a processor configured and arranged to communicate with the chemical biomarker sensor and to generate an adjustment to one or more of the stimulation parameters based on monitoring, measuring, or observation of the chemical biomarker by the chemical biomarker sensor. In at least some embodiments, the external programming unit includes the processor. In at least some embodiments, the processor is configured and arranged to perform the following actions: receive a sensor signal from the chemical biomarker sensor, wherein the sensor signal is generate by the chemical biomarker sensor in response to the monitoring, measuring, or observation of the chemical biomarker by the chemical biomarker sensor; and determine the adjustment to one or more stimulation parameters based on the sensor signal. In at least some embodiments the processor is configured and arranged to perform the additional following action: deliver the adjustment to one of the external programming unit or the control module.

In at least some embodiments, the system also includes a lead coupleable to the control module and including a plurality of electrodes for delivering the electrical stimulation signals to the patient tissue. In at least some embodiments, the adjustment is provided to the control module automatically and without user intervention. In at least some embodiments, the chemical biomarker sensor is an adenosine sensor.

Another embodiment is an electrical stimulation system including a chemical biomarker sensor configured and arranged to be disposed on or within a body of a patient and to monitor, measure, or observe a chemical biomarker; a control module configured and arranged to use stimulation parameters to provide electrical stimulation signals to an electrical stimulation lead coupled to the control module for stimulation of patient tissue; and a processor configured and arranged to communicate with the chemical biomarker sensor to generate an adjustment to one or more of the stimulation parameters based on the monitoring, measurement, or observation of the chemical biomarker by the chemical biomarker sensor.

In at least some embodiments, the control module includes the processor. In at least some embodiments, the control module is an implantable control module configured and arranged for implantation in a body of a patient, the implantable control module further including an antenna coupled to the processor. In at least some embodiments, the processor is external to the control module.

In at least some embodiments the processor is configured and arranged to perform the following actions: receive a sensor signal from the chemical biomarker sensor, wherein the sensor signal is generate by the chemical biomarker sensor in response to the monitoring, measuring, or observation of the chemical biomarker by the chemical biomarker sensor; and determine the adjustment to one or more stimulation parameters based on the sensor signal.

In at least some embodiments, the system also includes a lead coupleable to the control module and including a plurality of electrodes for delivering the electrical stimulation signals to the patient tissue. In at least some embodiments, the chemical biomarker sensor is disposed on the lead. In at least some embodiments, the chemical biomarker sensor is configured arranged for insertion into the patient and is external to the lead and the control module.

In at least some embodiments, the chemical biomarker sensor is disposed on the control module. In at least some embodiments, the chemical biomarker sensor is configured and arranged disposition external to the patient for monitoring, measuring, or observation of the chemical biomarker. In at least some embodiments, the chemical biomarker sensor is an adenosine sensor.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic block diagram of one embodiment of an electrical stimulation system with a chemical biomarker sensor, according to the invention;

FIG. 2 is a schematic block diagram of another embodiment of an electrical stimulation system with a chemical biomarker sensor, according to the invention;

FIG. 3 is a schematic block diagram of another embodiment of an electrical stimulation system with a chemical biomarker sensor, according to the invention;

FIG. 4 is a schematic block diagram of one embodiment of an external programming unit, according to the invention;

FIG. 5 is a schematic block diagram of one embodiment of a processing unit, according to the invention;

FIG. 6 is a flowchart of one embodiment of a method for adjusting stimulation parameters using a chemical biomarker sensor, according to the invention;

FIG. 7 is a flowchart of a second embodiment of a method for adjusting stimulation parameters using a chemical biomarker sensor, according to the invention;

FIG. 8 is a flowchart of a third embodiment of a method for adjusting stimulation parameters using a chemical biomarker sensor, according to the invention;

FIG. 9 is a schematic view of one embodiment of an electrical stimulation system that includes a paddle lead electrically coupled to a control module, according to the invention;

FIG. 10 is a schematic view of one embodiment of an electrical stimulation system that includes a percutaneous lead electrically coupled to a control module, according to the invention;

FIG. 11A is a schematic view of one embodiment of the control module of either FIG. 9 or FIG. 10 configured and arranged to electrically couple to an elongated device, according to the invention; and

FIG. 11B is a schematic view of one embodiment of a lead extension configured and arranged to electrically couple an elongated device to a control module, according to the invention.

### DETAILED DESCRIPTION

The present invention is directed to the area of implantable electrical stimulation systems and methods of making and using the systems. The present invention is also directed to implantable electrical stimulation systems that include devices or methods for electrical stimulation which utilize a chemical biomarker to provide feedback for adjusting stimulation parameters, as well as methods of making and using the electrical stimulation systems.

Suitable implantable electrical stimulation systems include, but are not limited to, a least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,949,395; 7,244,150; 7,672,734; 7,761,165; 7,974,706; 8,175,710; 8,224,450; and 8,364,278; and U.S. Patent Application Publication No. 2007/0150036, all of which are incorporated by reference.

Chemical biomarkers can be monitored, measured, or otherwise observed to provide feedback regarding the effect, efficacy, adequacy, or other results of electrical stimulation. The term “chemical biomarker” as used herein is directed to a chemical constituent (or combination of chemical constituents) of a biological system including, but not limited to, compounds, molecules, ions, elements, biomolecules, macromolecules, mixtures, solutions, and the like.

An electrical stimulation system can include one or more chemical biomarker sensors that can monitor, measure, or otherwise observe one or more chemical biomarkers and the response of the one or more chemical biomarkers when an electrical stimulation treatment is applied to the patient. The monitoring, measurement, or observation of the chemical biomarker may be directed to one or more properties of the chemical biomarker such as, for example, one or more of presence, absence, concentration, amount, increase in concentration or amount, decrease in concentration or amount, and the like, or any combination thereof.

Change in the one or more properties of the chemical biomarker can be indicative of one or more aspects of the electrical stimulation such as, for example, efficacy, increase or decrease in efficacy, desired treatment effect, increase or decrease in desired treatment effect, undesirable side-effect, increase or decrease in undesirable side-effect, or the like, or any combination thereof.

As an example, adenosine has been determined to be a chemical biomarker correlated with pain relief. Neuropathic analgesia has be linked to activation of the A3 receptor which occurs naturally with adenosine. Accordingly, the concentration or amount of adenosine, or changes in the concentration or amount of adenosine, can be correlated with the efficacy or effect of electrical stimulation (for example, in the spinal cord, brain, or other nerve or tissue) to treat pain.

The one or more chemical biomarker sensors can be implanted or inserted permanently (e.g., for at least one month, one year, five years, or longer) or temporarily (e.g., for no more than 1 minute, 5 minutes, 15 minutes, 30 minutes, 1 hour, 2 hours, 4 hours, six hours, twelve hours, or 1 day) in the patient, or can be disposed on the skin of the patient, or can be external to the patient, or (in the case of multiple chemical biomarker sensors) any combination thereof. If multiple chemical biomarker sensors are used, the chemical biomarker sensors may be the same or different and may sense the same or different chemical biomarkers.

The one or more chemical biomarker sensors can be placed in any suitable location. For example, a chemical biomarker sensor may be placed at or near the site of stimulation. In some embodiments, the chemical biomarker sensor may be positioned on the electrical stimulation lead or the control module of the electrical stimulation system. In other embodiments, the chemical biomarker sensor may be separate from the electrical stimulation lead or control module, although, optionally, the sensor may be physically coupled to the lead or control module through tether that optionally includes a conductor to carry signals from the sensor to the lead or control module.

As another example of sensor placement, a chemical biomarker sensor may be placed at or near a site at which an effect of the stimulation is expected. For example, electrical stimulation of the brain or spinal cord can produce effects in other parts of the body including other organs, limbs, nerves, tissues, or the like. As yet another example, the chemical biomarker sensor may be placed at or near a site where the chemical biomarker is produced, accumulated, used, or the like.

As indicated above, the chemical biomarker sensor may be implanted or inserted into the patient or can be placed on or near the skin of the patient. In some embodiments, the chemical biomarker sensor can be attached to the body of the patient using, for example, a band, cuff, belt, clamp, clip, friction, adhesive, or the like or any combination thereof.

In other embodiments, the chemical biomarker sensor can be external to the patient and the chemical biomarker sensor can observe the patient (or at least a portion of the patient) or body tissue or fluid may be transferred to the chemical biomarker sensor for evaluation. For example, the chemical biomarker sensor may be a laboratory/office instrument or a portable instrument, such as a meter, that a clinician or patient provides tissue or fluid for analysis. In some embodiments, the chemical biomarker sensor can be provided on, or attached to, an external programming unit (described in more detail below) or a patient remote control or a charging unit for the control module.

The monitoring, measurements, or observations from the chemical biomarker sensor can be used in a manual, automated, or semi-automated manner to alter one or more stimulation parameters to enhance the treatment. In at least some embodiments, the system may perform the monitoring, measurements, or observations under one or more conditions such as, for example, during a programming session; at regular or irregular intervals during operation of the system; or when initiated by a clinician, patient, or other individual; or any combination thereof.

The monitoring, measurements, or observations can be used to select one or more stimulation parameters or alter one or more stimulation parameters. The selection or alteration may be performed when initiated or on a periodic, irregular, or continuous basis.

In at least some embodiments, the electrical stimulation system can have a closed-loop feedback function using the one or more chemical biomarker sensors and the respective monitoring, measurements, or observations. The feedback function may be automated or semi-automated. In at least some embodiments, the feedback function may be initiated under one or more conditions such as, for example, during a programming session; at regular or irregular intervals during operation of the system; or when directed by a clinician, patient, or other individual.

In at least some embodiments, the monitoring, measurements, or observations from the chemical biomarker sensor can be used to steer the electrical stimulation (for example, the electrical current). Steering can be performed by, for example, altering the selection of electrode(s) that provide the electrical stimulation; altering the amplitude (or other stimulation parameters such as frequency or duration) of stimulation provided by given electrodes; or the like or any combination thereof. In at least some embodiments, steering of the electrical stimulation can include multiple timing channels which utilize the electrodes of the lead to generate different electric fields. The electric fields for the different timing channels can be interleaved temporally to alter the electrical stimulation of the patient tissue. Stimulation steering can be used to alter the electric field produced by the system and to alter the portion of patient tissue being stimulated or the amount of stimulation provided to a region of patient tissue. This can tailor the stimulation to the patient or to the current condition of the patient. Any combination of these steering methods can also be employed.

In at least some embodiments, one or more chemical biomarker sensors can be used to determine patient activity changes, changes in disease or disorder progression or modality, or changes in the stimulation system, or any combination thereof. The monitoring, measurements, or observations can be used to select or alter one or more electrical stimulation parameters. The system may perform the monitoring, measurements, or observations under one or more conditions such as, for example, during a programming session; at regular or irregular intervals during operation of the system; or when directed by a clinician, patient, or other individual. In at least some embodiments, the electrical stimulation system can have a closed-loop feedback function that allows the system to alter stimulation as a result of changes in patient activity, changes in the disease or disorder, or changes to the components of the system or their surroundings.

In addition to one or more chemical biomarker sensors, an electrical stimulation system can include one or more other sensors to measure biosignals. Information from those sensors can also be used to select or alter stimulation parameters. Systems that utilize such biosignal sensors are described in U.S. Provisional Patent Applications Ser. Nos. 62/028,704; 62/053,427; and 62/061,069, all of which are incorporated herein by reference. In the embodiments described herein that utilize one or more chemical biomarker sensors, it will be understood that such embodiments may also include one or more biosignal sensors.

An electrical stimulation system can include an implantable control module and one or more leads for delivering electrical energy from the control module to patient tissue. In other embodiments, the control module can be external and can deliver energy to implanted leads through a wired or wireless connection. Examples of systems with an external control module or external trial stimulator are described at U.S. patent application Ser. Nos. 14/325,176 and 14/330,330 and U.S. Provisional Patent Applications Ser. Nos. 62/012,141 and 62/053,589, all of which are incorporated herein by reference.

In yet other embodiments, the electrical stimulation system can be a transcutaneous electrical stimulation system (TENS) that uses electrodes positioned on or near patient skin to provide non-invasive therapy. Examples of TENS systems are described at U.S. Provisional Patent Application Ser. No. 62/053,501, incorporated herein by reference.

The discussion below uses an implantable electrical stimulation system with an implantable control module and one or more implantable leads for delivering therapy to a patient. It will be understood, however, that any of the other systems described herein (e.g., a TENS or external control module system) can be utilized with one or more chemical biomarker sensors in place of the implantable electrical stimulation system.

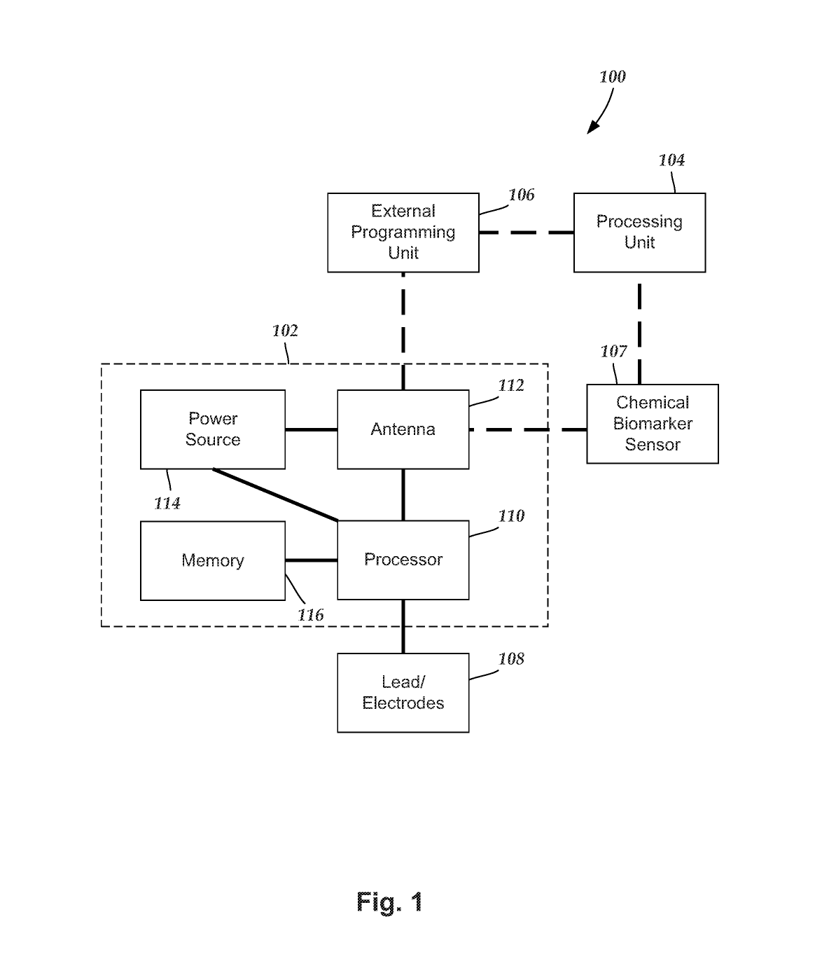

FIG. 1 illustrates schematically one embodiment of an electrical stimulation system 100 that includes an implantable control module (e.g., an implantable electrical stimulator or implantable pulse generator) 102, one or more leads 108 with electrodes, one or more external programming units 106, one or more chemical biomarker sensors 107, and a processing unit 104. Alternatively, the implantable control module 102 can be part of a microstimulator with the electrodes disposed on the housing of the microstimulator. The microstimulator may not include a lead or, in other embodiments, a lead may extend from the microstimulator. As yet another alternative, the control module can be external to the patient. One example of an external control module is an external trial stimulator that can be used temporarily during the implantation procedure to test stimulation using the lead.

It will be understood that the electrical stimulation system can include more, fewer, or different components and can have a variety of different configurations including those configurations disclosed in the references cited herein. For example, although FIG. 1 illustrates one external programming unit 106, one control module 102, and one chemical biomarker sensor 107, it will be understood that the system can include more than one external programming unit, more than one control module, more than one chemical biomarker sensor, or any combination thereof.

The lead 108 is coupled, or coupleable, to the implantable control module 102. The implantable control module 102 includes a processor 110, an antenna 112 (or other communications arrangement), a power source 114, and a memory 116, as illustrated in FIG. 1.

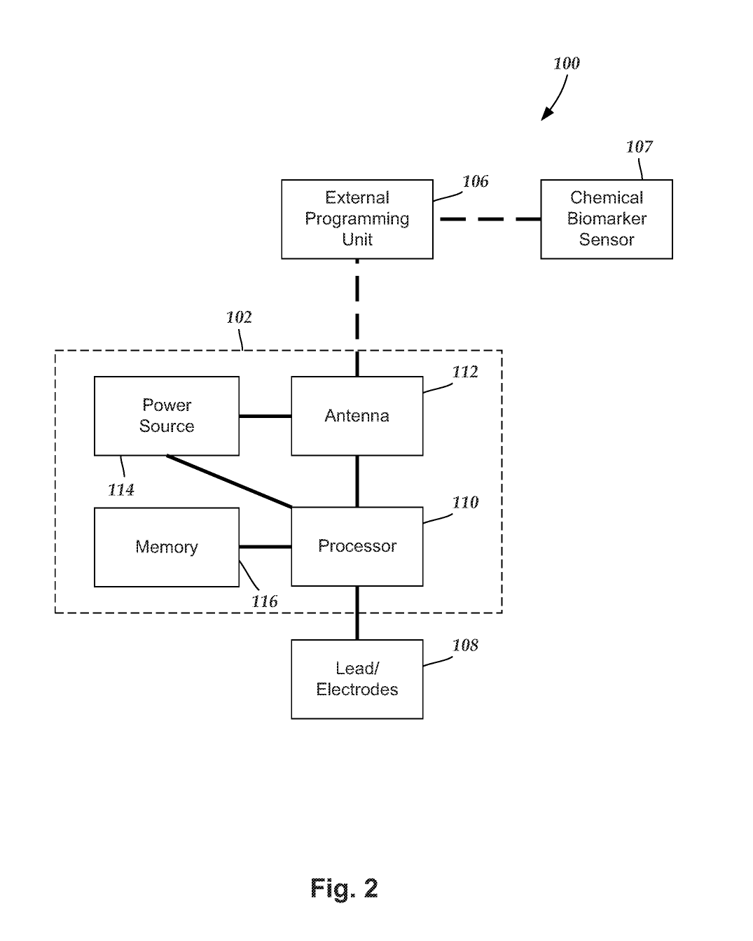

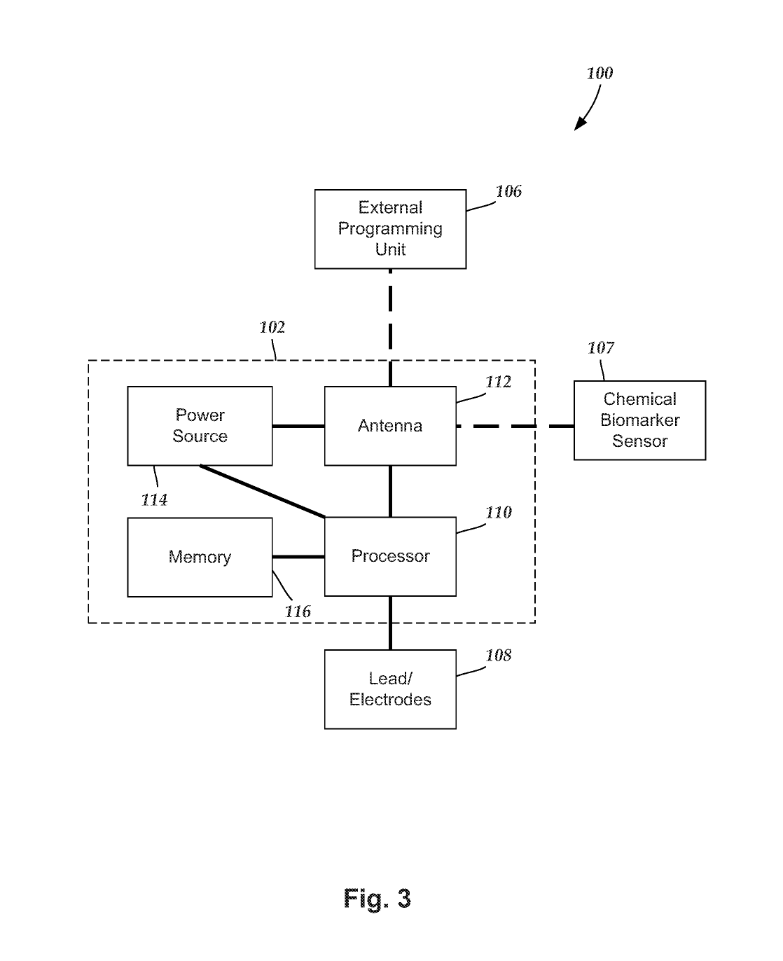

FIGS. 2 and 3 illustrate other embodiments in which the processing unit is omitted and the external programming unit 106, chemical biomarker sensor(s) 107, or control module 102 or any combination thereof can perform the functions of the processing unit. In the embodiment of FIG. 2, the chemical biomarker sensor 107 is in communication with the external programming unit 106. In the embodiment of FIG. 3, the chemical biomarker sensor 107 is in communication with the control module 102.

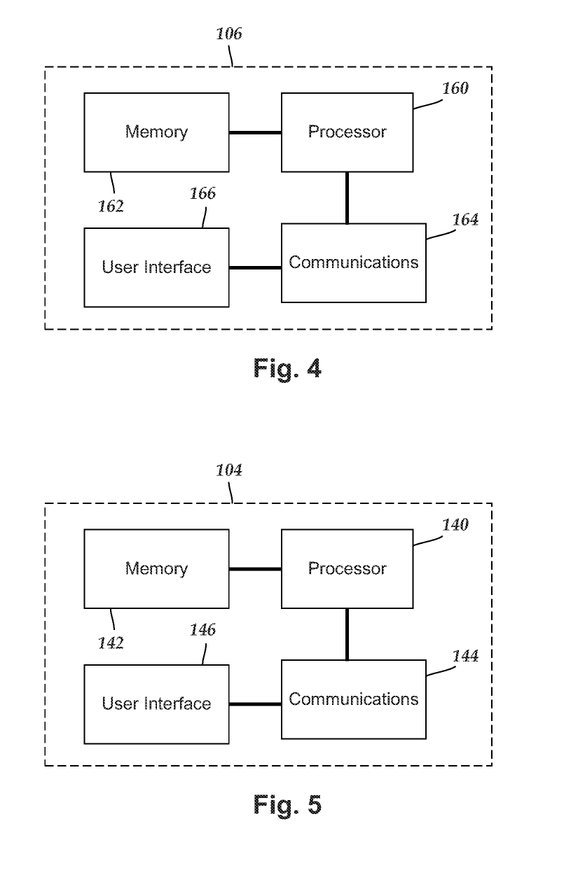

One example of an external programming unit 106 is illustrated in FIG. 4 and includes a processor 160, a memory 162, a communications arrangement 164 (such as an antenna or any other suitable communications device such as those described below), and a user interface 166. Suitable devices for use as an external programming unit can include, but are not limited to, a computer, a tablet, a mobile telephone, a personal desk assistant, a dedicated device for external programming, remote control, or the like. It will be understood that the external programming unit 106 can include a power supply or receive power from an external source or any combination thereof. The external programming unit 106 can be a home station or unit at a clinician's office or any other suitable device. In some embodiments, the external programming unit 106 can be a device that is worn on the skin of the user or can be carried by the user and can have a form similar to a pager, cellular phone, or remote control, if desired. The external programming unit 106 can be any unit that can provide information to the control module 102. One example of a suitable external programming unit 106 is a computer operated by the clinician or patient to send signals to the control module 102. Another example is a mobile device or an application on a mobile device that can send signals to the control module 102

One example of a processing unit 104 is illustrated in FIG. 5 and includes a processor 140, a memory 142, a communications arrangement 144 (such as an antenna or any other suitable communications device such as those described below), and an optional user interface 146. Suitable devices for use as a processing unit can include, but are not limited to, a computer, a tablet, a server or server farm, or the like. It will be understood that the processing unit 104 can include a power supply or receive power from an external source or any combination thereof.

Methods of communication between devices or components of a system can include wired (including, but not limited to, USB, mini/micro USB, HDMI, and the like) or wireless (e.g., RF, optical, infrared, near field communication (NFC), Bluetooth™, or the like) communications methods or any combination thereof. By way of further example, communication methods can be performed using any type of communication media or any combination of communication media including, but not limited to, wired media such as twisted pair, coaxial cable, fiber optics, wave guides, and other wired media and wireless media such as acoustic, RF, optical, infrared, NFC, Bluetooth™ and other wireless media. These communication media can be used for communications units 144, 164 or as antenna 112 or as an alternative or supplement to antenna 112.

Turning to the control module 102, some of the components (for example, a power source 114, an antenna 112, and a processor 110) of the electrical stimulation system can be positioned on one or more circuit boards or similar carriers within a sealed housing of the control module (implantable pulse generator,) if desired. Any power source 114 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the antenna 112 or a secondary antenna. The external power source can be in a device that is mounted on the skin of the user or in a unit that is provided near the user on a permanent or periodic basis.

If the power source 114 is a rechargeable battery, the battery may be recharged using the antenna 112, if desired. Power can be provided to the battery for recharging by inductively coupling the battery through the antenna to a recharging unit external to the user.

A stimulation signal, such as electrical current in the form of electrical pulses, is emitted by the electrodes of the lead 108 (or a microstimulator) to stimulate neurons, nerve fibers, muscle fibers, or other body tissues near the electrical stimulation system. Examples of leads are described in more detail below. The processor 110 is generally included to control the timing and electrical characteristics of the electrical stimulation system. For example, the processor 110 can, if desired, control one or more of the timing, frequency, strength, duration, and waveform of the pulses. In addition, the processor 110 can select which electrodes can be used to provide stimulation, if desired. In some embodiments, the processor 110 selects which electrode(s) are cathodes and which electrode(s) are anodes. In some embodiments, the processor 110 is used to identify which electrodes provide the most useful stimulation of the desired tissue.

With respect to the control module 102, external programming unit 106, and database unit 104, any suitable processor 110, 140, 160 can be used in these devices. For the control module 102, the processor 110 is capable of receiving and interpreting instructions from an external programming unit 106 that, for example, allows modification of pulse characteristics. In the illustrated embodiment, the processor 110 is coupled to the antenna 112. This allows the processor 110 to receive instructions from the external programming unit 106 to, for example, direct the pulse characteristics and the selection of electrodes, if desired. The antenna 112, or any other antenna described herein, can have any suitable configuration including, but not limited to, a coil, looped, or loopless configuration, or the like. In one embodiment, the antenna 112 is capable of receiving signals (e.g., RF signals) from the external programming unit 106 or chemical biomarker sensor 107.

The signals sent to the processor 110 via the antenna 112 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the control module 102 to cease operation, to start operation, to start charging the battery, or to stop charging the battery.

Optionally, the control module 102 may include a transmitter (not shown) coupled to the processor 110 and the antenna 112 for transmitting signals back to the external programming unit 106 or another unit capable of receiving the signals. For example, the control module 102 may transmit signals indicating whether the control module 102 is operating properly or not or indicating when the battery needs to be charged or the level of charge remaining in the battery. The processor 110 may also be capable of transmitting information about the pulse characteristics so that a user or clinician can determine or verify the characteristics.

Any suitable memory 116, 142, 162 can be used for the respective components of the system 100. The memory 116, 142, 162 illustrates a type of computer-readable media, namely computer-readable storage media. Computer-readable storage media may include, but is not limited to, nonvolatile, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

The user interface 166 of the external programming unit 106 and optional user interface 146 of the processing unit 104 can be, for example, a keyboard, mouse, touch screen, track ball, joystick, voice recognition system, or any combination thereof, and the like. Alternatively or additionally, the user interface 166 of the external programming unit 106 can include one or more microphones, sensors, cameras, or the like to obtain clinician or patient input. For example, the clinician or patient may provide input verbally (e.g. voice command recognition, voice recordings) or visually (e.g. video of patient, non-touch gesture recognition, or the like). In at least some embodiments, patient feedback can be provided by the clinician or other user through the external programming unit 106.

The one or more chemical biomarker sensors 107 can be any suitable chemical biomarker sensors including chemical biomarker sensors that utilize, for example, chemical, light (visible, infrared, ultraviolet, or any other spectrum of light), thermal, electrical, magnetic, or other methods (or any combination thereof) for monitoring or making measurements or observations.

The chemical biomarker sensor 107 can be in communication with the external programming unit 106, the control module 102, the processing unit 104, or any combination thereof. Such communication can be wired or wireless or any combination thereof using any of the communication methods described above. In at least some embodiments, the chemical biomarker sensor 107 can include a processor, a memory, or both.

In at least some embodiments, the chemical biomarker sensor 107 is deployed and used only during a programming session. In other embodiments, the chemical biomarker sensor 107 may be deployed on or within the patient for an extended period of time (for example, at least one day, one week, one month, six months, one year, or longer). In at least some embodiments, the chemical biomarker sensor 107 may be in regular or constant communication with the control module 102 or external programming unit 106. In at least some embodiments, the chemical biomarker sensor 107 may contact the control module 102, external programming unit 106, or processing unit 104 when requested, when a change in the chemical biomarker exceeds or falls below a threshold, at regular or irregular intervals, or any combination thereof.

In at least some embodiments, the monitoring, measurements, or observations from the chemical biomarker sensor(s) 107 are provided to the processing unit 104. The processing unit 104 includes an algorithm or other computer program that utilizes the chemical biomarker sensor monitoring, measurements, or observations and the current stimulation parameters and, optionally, other information regarding the patient, disease or disorder, and the like to determine adjustment to one or more of the stimulation parameters. The processing unit 104 can communicate the adjustment to a clinician or other user or to the external programming unit 106 or control module 102.

In other embodiments, the external programming unit 106 or control module 102 receives the monitoring, measurements, or observations from the chemical biomarker sensor(s) 107 and includes an algorithm or other computer program that utilizes the monitoring, measurements, or observations and the current stimulation parameters and, optionally, other information regarding the patient, disease or disorder, and the like to determine adjustment to one or more of the stimulation parameters. In yet other embodiments, the chemical biomarker sensor includes the algorithm or other computer program that determines adjustment to one or more of the stimulation parameters based on the monitoring, measurements, or observations of the chemical biomarker sensor.

In addition to the monitoring, measurements, or observations of the chemical biomarker sensor, the algorithm or computer program also receives the current stimulation parameters from, for example, the external programming unit or the control module or any other suitable source. The system can also incorporate one or more of medication information, demographics (for example, age, gender, ethnicity, height, weight, or the like), disease-specific details (for example, pain etiology(ies), number of prior back surgeries, relevant diagnoses, imaging findings, or the like) in the information used to determine adjustments to the stimulation parameters. The algorithm or computer program may determine adjustments based on patient-specific response to previous adjustments, based on population response to previous adjustments, based on patient activity or disease/disorder status determined from the monitoring, measurements, or observations of the chemical biomarker sensor, or any combination thereof.

In at least some embodiments, the system may utilize a step-wise methodology to altering the stimulation parameters. For example, the system may alter one or more stimulation parameters based on the monitoring, measurements, or observations of the chemical biomarker sensor and then observe the results of the alteration as measured using the chemical biomarker sensor (or based on other input such as patient or clinician feedback.) In at least some embodiments, the system waits for a latency period to allow the clinical effect to be measurable by the chemical biomarker sensor.

In some embodiments, the system may have the objective of improving or optimizing stimulation to produce a desired sensed clinical effect or may improve or co-optimize multiple sensed clinical effects or may improve or co-optimize one or more clinical effects and energy usage. Any suitable algorithmic technique can be used including, but not limited to, brute force parameter space searching, gradient search methods, genetic or stimulated annealing methods, machine learning or support vector machine methods, or the like. Such general techniques for algorithms are known.

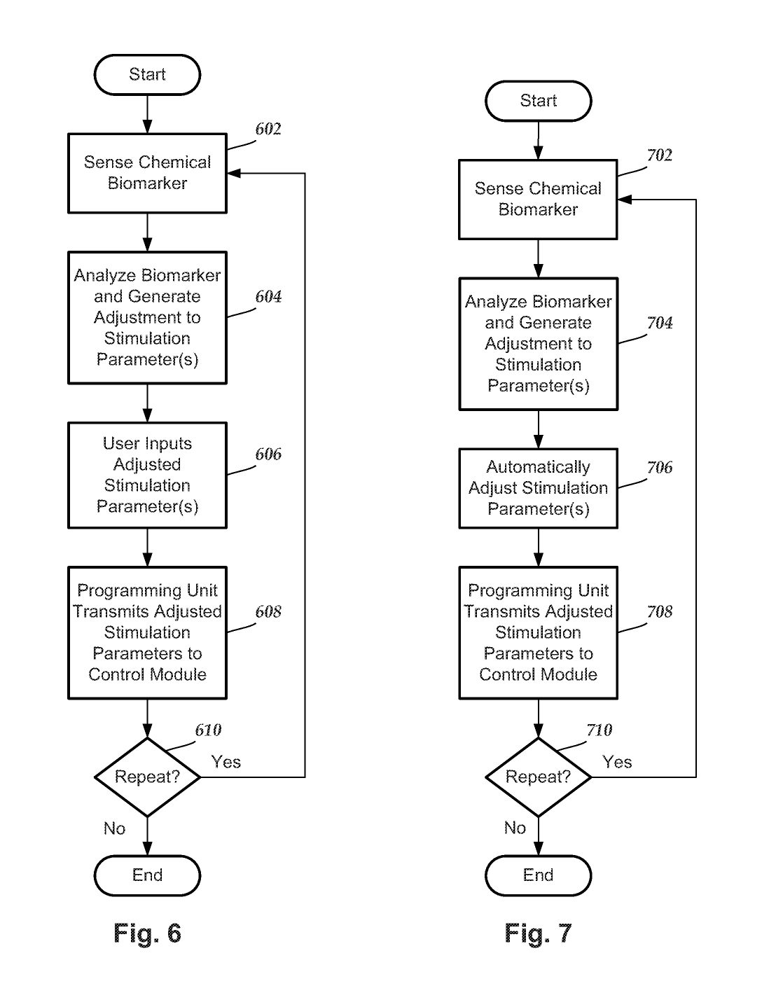

FIG. 6 is a flowchart of one embodiment of a method of adjusting stimulation parameters. In step 602, a chemical biomarker is sensed by one or more chemical biomarker sensors. In some embodiments, more than one chemical biomarker can be sensed or chemical biomarkers from two or more locations on the body of the patient can be sensed.

In step 604, the chemical biomarker is analyzed and an adjustment to one or more stimulation parameters is generated. Examples of stimulation parameters that can be adjusted include, but are not limited to, pulse frequency, pulse width, electrode field selection (anodes and cathodes which may can also affect the location of stimulation), pulse amplitude, pulse burst frequency or duration, pulse patterns, other pulse timing parameters, and the like. The analysis and generation of the adjustment can be performed by the processing unit 104, external programming unit 106, control module 102, or chemical biomarker sensor 107, or any combination thereof. The monitoring, measurement, or observation of chemical biomarker by the chemical biomarker sensor 107 can be communicated as a sensor signal to the processing unit 104, external programming unit 106, control module 102, or any combination thereof. In some embodiments, the sensor signal can be the signal as sensed by the chemical biomarker sensor. In other embodiments, the chemical biomarker sensor can process the monitoring, measurement, or observation to generate the sensor signal.

In step 606, a user (such as a clinician or patient) inputs the adjusted stimulation parameter(s) into the external programming unit 106. This process is semi-automated because it includes participation by the user. This participation may be desirable to provide user analysis of the adjusted stimulation parameters. This procedure may be useful, for example, during a control module programming session with a clinician. In such a procedure, the patient may also provide feedback regarding the adjusted stimulation.

In step 608, the external programming unit 106 transmits the adjusted stimulation parameters to the control module 102. The control module 102 then proceeds to deliver electrical stimulation using the adjusted stimulation parameters.

In step, 610, it is determined whether to repeat the process. If so, steps 602-610 are repeated. If not, the method terminates. In some embodiments, the process will automatically repeat without any formal decision to do so. In some embodiments, the process may repeat at regular or irregular intervals.

FIG. 7 is a flowchart of another embodiment of a method of adjusting stimulation parameters. In step 702, a chemical biomarker is sensed by one or more chemical biomarker sensors. In some embodiments, more than one chemical biomarker can be sensed or chemical biomarkers from two or more locations on the body of the patient can be sensed.

In step 704, the chemical biomarker is analyzed and an adjustment to one or more stimulation parameters is generated. Examples of stimulation parameters that can be adjusted include, but are not limited to, pulse frequency, pulse width, electrode field selection (anodes and cathodes which may can also affect the location of stimulation), pulse amplitude, pulse burst frequency or duration, pulse patterns, other pulse timing parameters, and the like. The analysis and generation of the adjustment can be performed by the processing unit 104, external programming unit 106, control module 102, or chemical biomarker sensor 107 or any combination thereof. The monitoring, measurement, or observation of chemical biomarker by the chemical biomarker sensor 107 can be communicated as a sensor signal to the processing unit 104, external programming unit 106, control module 102, or any combination thereof. In some embodiments, the sensor signal can be the signal as sensed by the chemical biomarker sensor. In other embodiments, the chemical biomarker sensor can process the monitoring, measurement, or observation to generate the sensor signal.

In step 706, the stimulation parameter(s) are automatically adjusted at the external programming unit 106. In some embodiments, such as during a control module programming session, the external programming unit 106 may optionally display the adjusted parameters so that the clinician or patient can halt the process, if desired, or observe or direct the path of tested parameters.

In step 708, the external programming unit 106 transmits the adjusted stimulation parameters to the control module 102. The control module 102 then proceeds to deliver electrical stimulation using the adjusted stimulation parameters.

In step, 710, it is determined whether to repeat the process. If so, steps 702-710 are repeated. If not, the method terminates. In some embodiments, the process will automatically repeat without any formal decision to do so. In some embodiments, the process may repeat at regular or irregular intervals.

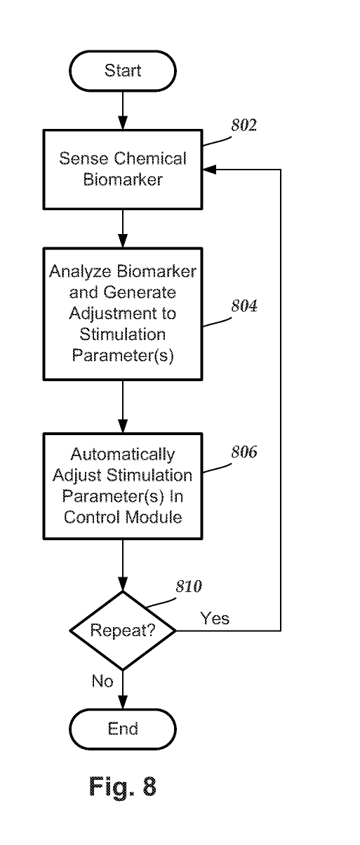

FIG. 8 is a flowchart of another embodiment of a method of adjusting stimulation parameters. In step 802, a chemical biomarker is sensed by one or more chemical biomarker sensors. In some embodiments, more than one chemical biomarker can be sensed or chemical biomarkers from two or more locations on the body of the patient can be sensed.

In step 804, the chemical biomarker is analyzed and an adjustment to one or more stimulation parameters is generated. Examples of stimulation parameters that can be adjusted include, but are not limited to, pulse frequency, pulse width, electrode field selection (anodes and cathodes which may can also affect the location of stimulation), pulse amplitude, pulse burst frequency or duration, pulse patterns, other pulse timing parameters, and the like. The analysis and generation of the adjustment can be performed by the processing unit 104, external programming unit 106, control module 102, or chemical biomarker sensor 107 or any combination thereof. The monitoring, measurement, or observation of chemical biomarker by the chemical biomarker sensor 107 can be communicated as a sensor signal to the processing unit 104, external programming unit 106, control module 102, or any combination thereof. In some embodiments, the sensor signal can be the signal as sensed by the chemical biomarker sensor. In other embodiments, the chemical biomarker sensor can process the monitoring, measurement, or observation to generate the sensor signal.

In step 806, the stimulation parameters are automatically adjusted in the control module 102. The control module 102 then proceeds to deliver electrical stimulation using the adjusted stimulation parameters. This process may be particularly useful where the control module 102 receives the monitoring, measurements, or observations or the adjustment to the stimulation parameters directly from the chemical biomarker sensor 107.

In step, 810, it is determined whether to repeat the process. If so, steps 802-810 are repeated. If not, the method terminates. In some embodiments, the process will automatically repeat without any formal decision to do so. In some embodiments, the process may repeat at regular or irregular intervals.

The processes illustrated in FIGS. 6-8 can be used as a feedback loop to adjust stimulation parameters. The feedback loop may be part of a programming session. Alternatively or additionally, the electrical stimulation system may initiate the feedback loop on a regular or irregular basis or when requested by a user, clinician, or other individual to adjust stimulation parameters.

It will be understood that the system can include one or more of the methods described hereinabove with respect to FIGS. 6-8 in any combination. The methods, systems, and units described herein may be embodied in many different forms and should not be construed as limited to the embodiments set forth herein. Accordingly, the methods, systems, and units described herein may take the form of an entirely hardware embodiment, an entirely software embodiment or an embodiment combining software and hardware aspects. The methods described herein can be performed using any type of processor or any combination of processors where each processor performs at least part of the process.

It will be understood that each block of the flowchart illustrations, and combinations of blocks in the flowchart illustrations and methods disclosed herein, can be implemented by computer program instructions. These program instructions may be provided to a processor to produce a machine, such that the instructions, which execute on the processor, create means for implementing the actions specified in the flowchart block or blocks or described for the control modules, external programming units, sensors, systems and methods disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

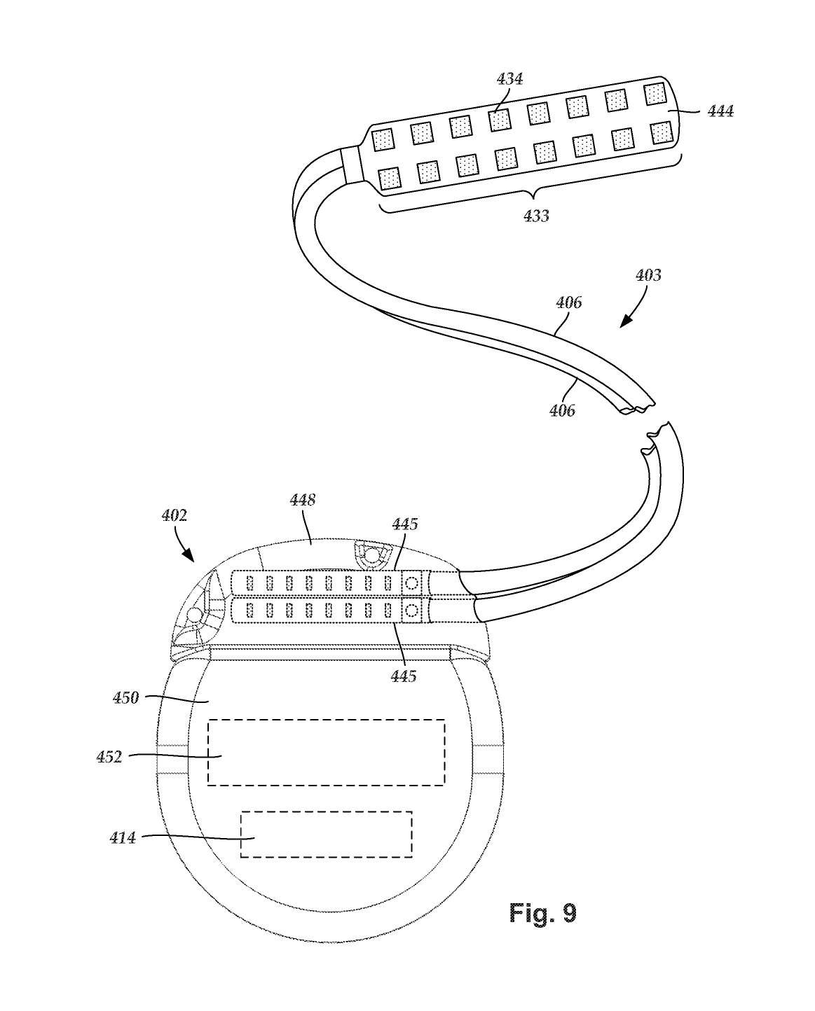

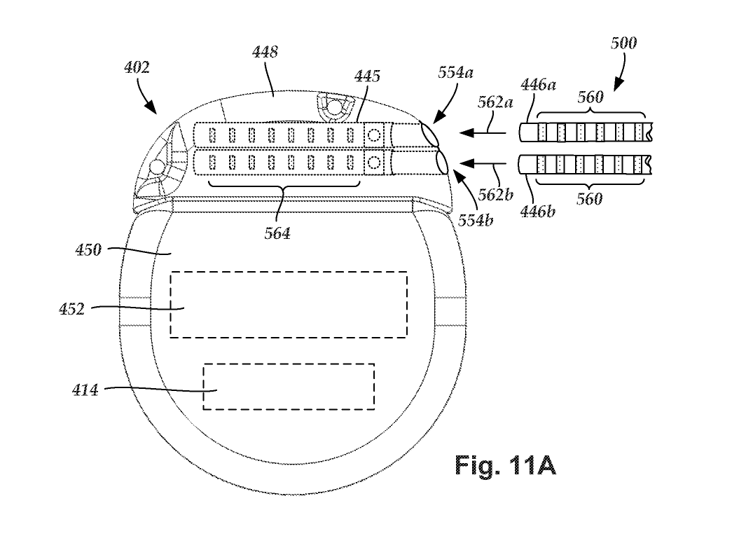

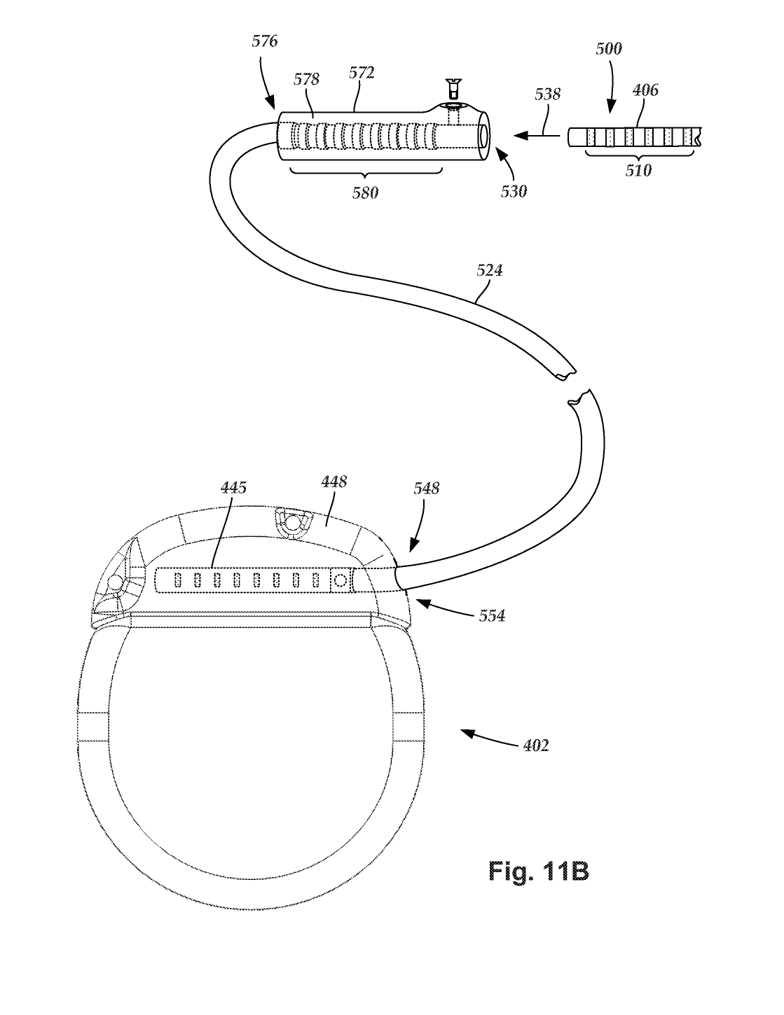

FIG. 9 illustrates one embodiment of a control module 402 and lead 403. The lead 403 includes a paddle body 444 and one or more lead bodies 446. In FIG. 9, the lead 403 is shown having two lead bodies 446. It will be understood that the lead 403 can include any suitable number of lead bodies including, for example, one, two, three, four, five, six, seven, eight or more lead bodies 446. An array of electrodes 433, such as electrode 434, is disposed on the paddle body 444, and one or more terminals (e.g., 560 in FIGS. 11A and 11B) are disposed along each of the one or more lead bodies 446. In at least some embodiments, the lead has more electrodes than terminals.

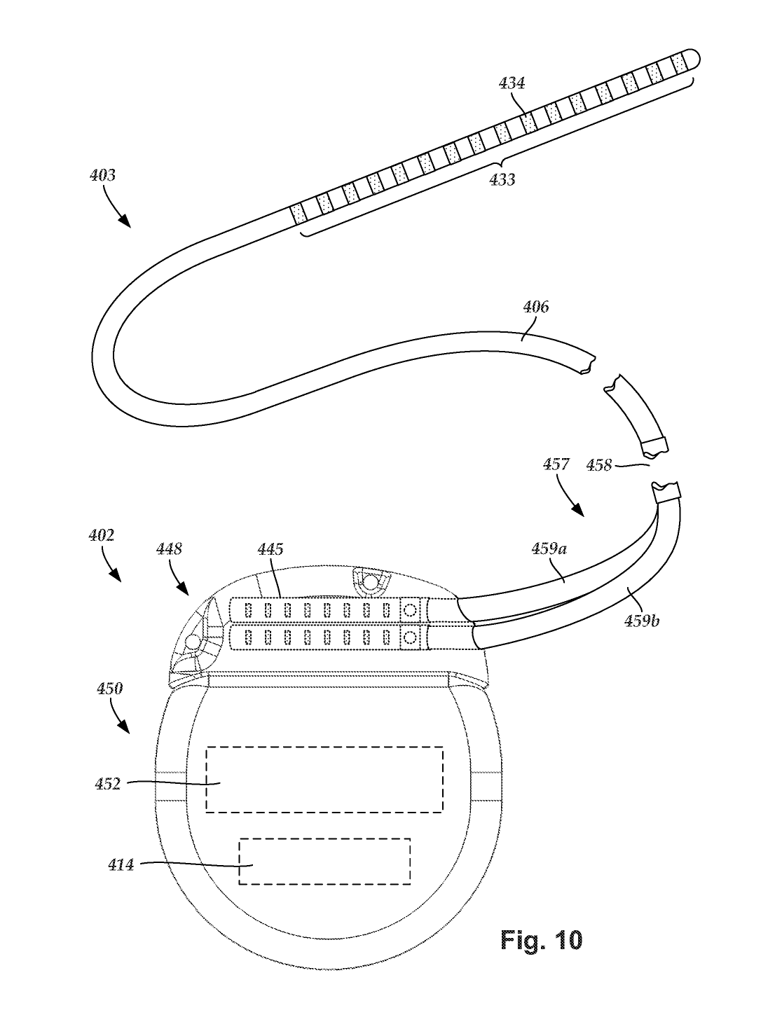

FIG. 10 illustrates schematically another embodiment in which the lead 403 is a percutaneous lead. In FIG. 10, the electrodes 434 are shown disposed along the one or more lead bodies 446. In at least some embodiments, the lead 403 is isodiametric along a longitudinal length of the lead body 446.

The lead 403 can be coupled to the implantable control module 402 in any suitable manner. In FIG. 9, the lead 403 is shown coupling directly to the implantable control module 402. In at least some other embodiments, the lead 403 couples to the implantable control module 402 via one or more intermediate devices (500 in FIGS. 11A and 11B). For example, in at least some embodiments one or more lead extensions 524 (see e.g., FIG. 11B) can be disposed between the lead 403 and the implantable control module 402 to extend the distance between the lead 403 and the implantable control module 402. Other intermediate devices may be used in addition to, or in lieu of, one or more lead extensions including, for example, a splitter, an adaptor, or the like or combinations thereof. It will be understood that, in the case where the electrical stimulation system includes multiple elongated devices disposed between the lead 403 and the implantable control module 402, the intermediate devices may be configured into any suitable arrangement.

In FIG. 10, the electrical stimulation system 400 is shown having a splitter 457 configured and arranged for facilitating coupling of the lead 403 to the implantable control module 402. The splitter 457 includes a splitter connector 458 configured to couple to a proximal end of the lead 403, and one or more splitter tails 459a and 459b configured and arranged to couple to the implantable control module 402 (or another splitter, a lead extension, an adaptor, or the like).

The implantable control module 402 includes a connector housing 448 and a sealed electronics housing 450. An electronic subassembly 452 (which includes the processor 110 (see, FIGS. 1-3) and the power source 414 are disposed in the electronics housing 450. A connector 445 is disposed in the connector housing 448. The connector 445 is configured and arranged to make an electrical connection between the lead 403 and the electronic subassembly 452 of the implantable control module 402.

The electrical stimulation system or components of the electrical stimulation system, including the paddle body 444, the one or more of the lead bodies 446, and the implantable control module 402, are typically implanted into the body of a patient. The electrical stimulation system can be used for a variety of applications including, but not limited to deep brain stimulation, neural stimulation, spinal cord stimulation, muscle stimulation, and the like.

The electrodes 434 can be formed using any conductive, biocompatible material. Examples of suitable materials include metals, alloys, conductive polymers, conductive carbon, and the like, as well as combinations thereof. In at least some embodiments, one or more of the electrodes 434 are formed from one or more of: platinum, platinum iridium, palladium, palladium rhodium, or titanium.

Any suitable number of electrodes 434 can be disposed on the lead including, for example, four, five, six, seven, eight, nine, ten, eleven, twelve, fourteen, sixteen, twenty-four, thirty-two, or more electrodes 434. In the case of paddle leads, the electrodes 434 can be disposed on the paddle body 444 in any suitable arrangement. In FIG. 9, the electrodes 434 are arranged into two columns, where each column has eight electrodes 434.

The electrodes of the paddle body 444 (or one or more lead bodies 446) are typically disposed in, or separated by, a non-conductive, biocompatible material such as, for example, silicone, polyurethane, polyetheretherketone (“PEEK”), epoxy, and the like or combinations thereof. The one or more lead bodies 446 and, if applicable, the paddle body 444 may be formed in the desired shape by any process including, for example, molding (including injection molding), casting, and the like. The non-conductive material typically extends from the distal ends of the one or more lead bodies 446 to the proximal end of each of the one or more lead bodies 446.

In the case of paddle leads, the non-conductive material typically extends from the paddle body 444 to the proximal end of each of the one or more lead bodies 446. Additionally, the non-conductive, biocompatible material of the paddle body 444 and the one or more lead bodies 446 may be the same or different. Moreover, the paddle body 444 and the one or more lead bodies 446 may be a unitary structure or can be formed as two separate structures that are permanently or detachably coupled together.

One or more terminals (e.g., 560 in FIGS. 11A-11B) are typically disposed along the proximal end of the one or more lead bodies 446 of the electrical stimulation system 400 (as well as any splitters, lead extensions, adaptors, or the like) for electrical connection to corresponding connector contacts (e.g., 564 in FIGS. 11A-11B). The connector contacts are disposed in connectors (e.g., 445 in FIGS. 9-11B; and 572 FIG. 11B) which, in turn, are disposed on, for example, the implantable control module 402 (or a lead extension, a splitter, an adaptor, or the like). One or more electrically conductive wires, cables, or the like (i.e., “conductors”—not shown) extend from the terminal(s) to the electrode(s). In at least some embodiments, there is at least one (or exactly one) terminal conductor for each terminal which extends to at least one (or exactly one) of the electrodes.

The one or more conductors are embedded in the non-conductive material of the lead body 446 or can be disposed in one or more lumens (not shown) extending along the lead body 446. For example, any of the conductors may extend distally along the lead body 446 from the terminals 560.

FIG. 11A is a schematic side view of one embodiment of a proximal end of one or more elongated devices 500 configured and arranged for coupling to one embodiment of the connector 445. The one or more elongated devices may include, for example, one or more of the lead bodies 446 of FIG. 9, one or more intermediate devices (e.g., a splitter, the lead extension 524 of FIG. 11B, an adaptor, or the like or combinations thereof), or a combination thereof.

The connector 445 defines at least one port into which a proximal ends 446A, 446B of the elongated device 500 can be inserted, as shown by directional arrows 562a, 562b. In FIG. 11A (and in other figures), the connector housing 448 is shown having two ports 554a, 554b. The connector housing 448 can define any suitable number of ports including, for example, one, two, three, four, five, six, seven, eight, or more ports.

The connector 445 also includes one or more connector contacts, such as connector contact 564, disposed within each port 554a, 554b. When the elongated device 500 is inserted into the ports 554a, 554b, the connector contact(s) 564 can be aligned with the terminal(s) 560 disposed along the proximal end(s) of the elongated device(s) 500 to electrically couple the implantable control module 402 to the electrodes (434 of FIG. 9) disposed on the paddle body 445 of the lead 403. Examples of connectors in implantable control modules are found in, for example, U.S. Pat. Nos. 7,244,150 and 8,224,450, which are incorporated by reference.

FIG. 11B is a schematic side view of another embodiment that includes a lead extension 524 that is configured and arranged to couple one or more elongated devices 500 (e.g., one of the lead bodies 446 of FIGS. 9 and 10, the splitter 457 of FIG. 10, an adaptor, another lead extension, or the like or combinations thereof) to the implantable control module 402. In FIG. 11B, the lead extension 524 is shown coupled to a single port 554 defined in the connector 445. Additionally, the lead extension 524 is shown configured and arranged to couple to a single elongated device 500. In alternate embodiments, the lead extension 524 is configured and arranged to couple to multiple ports 554 defined in the connector 445, or to receive multiple elongated devices 500, or both.

A lead extension connector 572 is disposed on the lead extension 524. In FIG. 11B, the lead extension connector 572 is shown disposed at a distal end 576 of the lead extension 524. The lead extension connector 572 includes a connector housing 578. The connector housing 578 defines at least one port 530 into which terminal(s) 560 of the elongated device 500 can be inserted, as shown by directional arrow 538. The connector housing 578 also includes a plurality of connector contacts, such as connector contact 580. When the elongated device 500 is inserted into the port 530, the connector contacts 580 disposed in the connector housing 578 can be aligned with the terminal(s) 560 of the elongated device 500 to electrically couple the lead extension 524 to the electrodes (434 of FIGS. 9 and 10) disposed along the lead (403 in FIGS. 9 and 10).

In at least some embodiments, the proximal end of the lead extension 524 is similarly configured and arranged as a proximal end of the lead 403 (or other elongated device 500). The lead extension 524 may include one or more electrically conductive wires (not shown) that electrically couple the connector contact(s) 580 to a proximal end 548 of the lead extension 524 that is opposite to the distal end 576. The conductive wire(s) disposed in the lead extension 524 can be electrically coupled to one or more terminals (not shown) disposed along the proximal end 548 of the lead extension 524. The proximal end 548 of the lead extension 524 is configured and arranged for insertion into a connector disposed in another lead extension (or another intermediate device). As shown in FIG. 11B, the proximal end 548 of the lead extension 524 is configured and arranged for insertion into the connector 445.

The embodiments of FIGS. 9-11B illustrate a control module 402 with a connector 445 into which a proximal end portion of the lead or lead extension can be removably inserted. It will be recognized, however, that other embodiments of a control module and lead can have the lead or lead extension permanently attached to the control module. Such an arrangement can reduce the size of the control module as the conductors in the lead can be permanently attached to the electronic subassembly. It will also be recognized that, in at least some embodiments, more than one lead can be attached to a control module.

The above specification and examples provide a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. An electrical stimulation system, comprising:
an electrical stimulation lead configured and arranged for implantation in a body of a patient;
a control module coupleable to the electrical stimulation lead and comprising an antenna and a processor coupled to the antenna, wherein the control module is configured and arranged to provide electrical stimulation signals to the electrical stimulation lead for electrical stimulation of patient tissue;
an external programming unit configured and arranged to communicate with the processor of the implantable control module using the antenna and to provide or update stimulation parameters for production of the electrical stimulation signals; and
a chemical biomarker sensor configured and arranged to monitor, measure, or observe a chemical biomarker in response to the electrical stimulation.

2. The electrical stimulation system of claim 1, wherein the control module is configured and arranged for implantation in the body of the patient.

3. The electrical stimulation system of claim 1, further comprising a processor configured and arranged to communicate with the chemical biomarker sensor and to generate an adjustment to one or more of the stimulation parameters based on monitoring, measuring, or observation of the chemical biomarker by the chemical biomarker sensor.

4. The electrical stimulation system of claim 3, wherein the external programming unit comprises the processor.

5. The electrical stimulation system of claim 3, wherein the processor is configured and arranged to perform the following actions:
receive a sensor signal from the chemical biomarker sensor, wherein the sensor signal is generate by the chemical biomarker sensor in response to the monitoring, measuring, or observation of the chemical biomarker by the chemical biomarker sensor; and
determine the adjustment to one or more stimulation parameters based on the sensor signal.

6. The electrical stimulation system of claim 5, wherein the processor is configured and arranged to perform the additional following action:
deliver the adjustment to one of the external programming unit or the control module.

7. The electrical stimulation system of claim 1, further comprising a lead coupleable to the control module and comprising a plurality of electrodes for delivering the electrical stimulation signals to the patient tissue.

8. The electrical stimulation system of claim 1, wherein the adjustment is provided to the control module automatically and without user intervention.

9. The electrical stimulation system of claim 1, wherein the chemical biomarker sensor is an adenosine sensor.

10. An electrical stimulation system, comprising:
a chemical biomarker sensor configured and arranged to monitor, measure, or observe a chemical biomarker in a body of a patient;
a control module configured and arranged to use stimulation parameters to provide electrical stimulation signals to an electrical stimulation lead coupled to the control module for stimulation of patient tissue; and
a processor configured and arranged to communicate with the chemical biomarker sensor to generate an adjustment to one or more of the stimulation parameters based on the monitoring, measurement, or observation of the chemical biomarker by the chemical biomarker sensor.

11. The electrical stimulation system of claim 10, wherein the control module comprises the processor.

12. The electrical stimulation system of claim 10, wherein the control module is an implantable control module configured and arranged for implantation in a body of a patient, the implantable control module further comprising an antenna coupled to the processor.

13. The electrical stimulation system of claim 10, wherein the processor is external to the control module.

14. The electrical stimulation system of claim 10, wherein the processor is configured and arranged to perform the following actions:
receive a sensor signal from the chemical biomarker sensor, wherein the sensor signal is generate by the chemical biomarker sensor in response to the monitoring, measuring, or observation of the chemical biomarker by the chemical biomarker sensor; and
determine the adjustment to one or more stimulation parameters based on the sensor signal.

15. The electrical stimulation system of claim 10, further comprising a lead coupleable to the control module and comprising a plurality of electrodes for delivering the electrical stimulation signals to the patient tissue.

16. The electrical stimulation system of claim 15, wherein the chemical biomarker sensor is disposed on the lead.

17. The electrical stimulation system of claim 15, wherein the chemical biomarker sensor is configured arranged for insertion into the patient and is external to the lead and the control module.

18. The electrical stimulation system of claim 10, wherein the chemical biomarker sensor is disposed on the control module.

19. The electrical stimulation system of claim 10, wherein the chemical biomarker sensor is configured and arranged disposition external to the patient for monitoring, measuring, or observation of the chemical biomarker.

20. The electrical stimulation system of claim 10, wherein the chemical biomarker sensor is an adenosine sensor.
